(19) United States
(12) Patent Application Publication
(10) Pub. No.: US 2020/0230526 A1
(43) Pub. Date: Jul. 23, 2020

(54) METHODS OF PREPARING LIQUID EXCHANGED BIOLOGICAL FRACTIONS, AND DEVICES FOR USE IN PRACTICING THE SAME

(71) Applicant: ARTERIOCYTE MEDICAL SYSTEMS, INC., St . Louis, MO (US)

(72) Inventors: John Raymond CHAPMAN, Sacramento, CA (US); Rodney D. SPARKS, Sacramento, CA (US)

(21) Appl. No.: 16/633,131

(22) PCT Filed: Jul. 20, 2018
(86) PCT No.: PCT/US2018/043172

§ 371 Date: Jan. 22, 2020

Related U.S. Application Data
(60) Provisional application No. 62/536,359, filed on Jul. 24, 2017.

(51) Int. Cl. B01D 21/26; A61K 35/19; G01N 1/40; B01D 11/04

(52) U.S. Cl. CPC B01D 21/26; B01D 11/0492; G01N 1/4077; A61K 35/19

(57) ABSTRACT

Methods of preparing liquid exchanged biological fractions are provided. Aspects of the present disclosure according to certain embodiments include introducing a biological sample into a centrifugation container having a buoy, subjecting the biological sample to a force of centrifugation to produce two or more fractions in the biological sample, collecting a first fraction of the biological sample from the container and introducing a liquid exchange fluid into the container to produce a liquid exchanged fraction that is a mixture of the liquid exchange fluid and a second fraction of the biological sample. Compositions having a liquid exchanged platelet-rich fluid and kits for practicing embodiments of the subject methods are also provided. Methods for applying compositions having a liquid exchanged platelet-rich fluid to a body site of the subject are described.

### CROSS-REFERENCE TO RELATED APPLICATIONS

Pursuant to 35 U.S.C. § 119 (e), this application claims priority to the filing date of United States Provisional Patent Application No. 62/536,359, filed Jul. 24, 2017; the disclosure of which application is herein incorporated by reference.

### INTRODUCTION

Centrifugation has been used in the separation of components in a suspended medium to obtain cells, organelles or macromolecules contained in multi-component biologic fluids including bone marrow, whole blood, peripheral blood, urine, phlegm, synovial semen, milk, saliva, mucus, sputum, exudates, cerebrospinal fluid, amniotic fluid, cord blood, intestinal fluid, cell suspensions, tissue digests, tumor cell containing cell suspensions, microbe containing cell suspensions, radiolabelled cell suspensions and cell culture fluid which may or may not contain additional substances (e.g., anticoagulants to prevent clotting). Centrifugation of a medium having suspended particles causes the particles to sediment in the direction outward from the axis of rotation. The force generated by centrifugation is proportional to the speed of rotation and the radius of the rotor. At a fixed force and medium viscosity, the sedimentation rate of the particle is proportional to the molecular weight of the particle and the difference between its density and the density of the medium.

Components from biological fluids are used in a variety of therapeutic, diagnostic and research applications. Many biological fluid chemistry tests require separation of the components in the biological fluid. For example, when the biological fluid is blood, white blood cells, red blood cells, platelets and plasma components are often separated for testing. Enriched preparations of biological samples having sufficient concentration of components for the desired therapeutic, diagnostic or research use, can often require numerous and lengthy manipulations which often degrades the recovered materials or diminishes the amount of recoverable biological sample components. For example, multiple iterations of separation and washing of biological samples can be deleterious to components such as white blood cells, red blood cells and platelets due to over-processing.

### SUMMARY

Methods of preparing liquid exchanged biological fractions are provided. Aspects of the methods according to certain embodiments include introducing a biological sample into a centrifugation container having a buoy, subjecting the biological sample to a force of centrifugation to produce two or more fractions in the biological sample, collecting a first fraction of the biological sample from the container and introducing a liquid exchange fluid into the container to produce a liquid exchanged fraction that is a mixture of the liquid exchange fluid and a second fraction of the biological sample. In some embodiments, the biological sample is subjected to a single centrifugation step. In some instances, 75% by volume or more of the first fraction is removed from the container. In other instances, 90% by volume or more of the first fraction is removed from the container.

In some embodiments, the method includes positioning the centrifugation container at a first angle with respect to an axis orthogonal to the ground, removing the first fraction of the biological sample from the container, maintaining the centrifugation container at the first angle and introducing the liquid exchange fluid into the container, mixing the liquid exchange fluid with the second fraction of the biological sample in the buoy; and removing the liquid exchanged fraction from the container. In some instances, the liquid exchange fluid is mixed with the second fraction in the buoy. In certain instances, methods include re-introducing the liquid exchanged fraction into the container to rinse the buoy. This may be repeated 2 or more times, such as 5 or more times. In certain embodiments, positioning the centrifugation container at the first angle includes positioning the centrifugation container in an adjustable tilter stand and tilting the centrifugation container in the adjustable tilter stand to the first angle. In embodiments, the angle may range, such as from 5° to 45° with respect to an axis orthogonal to the ground.

In embodiments, the biological sample may be whole blood, anticoagulated whole blood, bone marrow aspirate, anticoagulated bone marrow aspirate, adipose derived cells, stromal vascular fraction and a combination thereof. In some embodiments, the biological sample is a blood sample. In other embodiments, the biological sample is a bone marrow aspirate. In still other embodiments, the biological sample is an adipose tissue containing sample. Where the biological sample is a blood sample, in certain instances, the first fraction includes platelet poor plasma and the second fraction includes platelets and white blood cells. In these embodiments, the liquid exchanged fraction is a composition that includes a liquid exchanged platelet-rich fluid.

The liquid exchange fluid is, in certain embodiments, an aqueous composition. In some instances, the liquid exchange fluid includes one or more of electrolytes, proteins, activating compounds, ions as well as other active agents. In some embodiments, the liquid exchange fluid includes a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof. In certain embodiments, the liquid exchange fluid is a buffered saline. The liquid exchange fluid in some instances has a citrate ion concentration that is 10 mM or less, such as 5 mM or less, such as 1 mM or less. In certain instance, the liquid exchange fluid does not have any citrate ions (i.e., a citrate ion concentration of 0 mM)

In certain embodiments of the present disclosure, the sample is a biological sample (e.g., blood or bone marrow aspirate) and methods include introducing a biological sample into the container of a separation device having a buoy configured to be displaced along a longitudinal axis within the container, subjecting the blood sample to a force of centrifugation to produce two or more fractions in the biological sample, each fraction having a biological component of a different density and collecting one or more of the separated components, then introducing a new fluid, e.g., a wash fluid, into the device and then collecting one or more of the separated components.

In some instances, subjecting the biological sample to a force of centrifugation is sufficient to displace the buoy proximally along a longitudinal axis within the container from the bottom of the container to a position at the interface between a first fraction and a second fraction of the biological sample. In certain embodiments, collecting one or more components of the biological sample includes removing a portion or preferably all of a first separated fraction of the biological sample, introducing a new cell resuspending fluid (cell washing fluid) into the device, mixing the new cell resuspending fluid with a second separated fraction within the buoy to produce a mixture of the new resuspending fluid and the second separated fraction, i.e., a liquid exchanged biological fraction, and removing the resultant mixture from the container.

In other embodiments, the present invention provides a means of collecting varying volumes of one or more components of the sample by a step comprising positioning the container of the separation device at a first angle (e.g., 20 degrees or more) with respect to an axis orthogonal to the ground, removing a portion of a first separated fraction of the sample through a conduit which extends from a port in the cap to the proximal end of the buoy, rotating the container by a second angle (e.g., 180 degrees) along the longitudinal axis of the container, aspirating the remaining portion of the first separated fraction of the sample through the conduit, without mixing the remaining portion of the first separated fraction with a second separated fraction within the buoy, introducing through said conduit a new cell resuspending fluid, mixing the remaining new resuspending fluid with said second separated fraction within the buoy, to produce a mixture of the new resuspending fluid and the second separated fraction and removing the resultant mixture, i.e., liquid exchanged biological fraction from the container.

In some instances, the first fraction contains platelet poor plasma and the second fraction contains white blood cells and platelet rich plasma and the new resuspending fluid (e.g., cell washing fluid) can be any fluid suitable for injection into a mammalian body.

In still other embodiments, the present disclosure provides methods of cell washing by collecting varying volumes of one or more components of the sample by a step that includes positioning the previously centrifuged device at a first angle with respect to an axis orthogonal to the ground (e.g., in a tilter stand); removing substantially all (e.g., >90%) of a first fraction of the biological sample; introducing a cell washing fluid without tilting the device to a second angular position with respect to the axis orthogonal to the ground; mixing the cell washing fluid with a second fraction of the biological fluid to produce a mixture of the second fraction and the cell washing fluid; and removing the resultant mixture, e.g., liquid exchanged biological fraction, from the container.

In some instances, the first fraction contains platelet poor plasma and the second fraction contains white blood cells and platelets and the wash fluid is any fluid suitable for injection into a mammalian body. When the wash fluid is saline and the second fraction contains white blood cells and platelets the resulting mixture is platelet rich saline.

Aspects of the present disclosure also include kits for practicing the subject methods. Kits according to certain embodiments include a liquid exchange fluid and a centrifugation container having a buoy configured to be displaced along a longitudinal axis within the container. In some embodiments, the kit also includes one or more syringes for removing fractions from the centrifuge container after subjecting a biological sample to a force of centrifugation. In some instances, the kit includes an adjustable tilter stand for positioning the centrifugation container at a plurality of angles with respect to an axis orthogonal to the ground. In certain instances, kits include instructions for practicing the subject methods, such as to position the centrifugation container at an angle with respect to an axis orthogonal to the ground, remove a first fraction of a biological sample from the container, maintain the centrifugation container at the angle and introduce the liquid exchange fluid into the container, mix the liquid exchange fluid with a second fraction of the biological sample in the buoy and remove the liquid exchanged fraction from the container. In these embodiments, the instructions may specify that the first angle is from 5° to 45° with respect to an axis orthogonal to the ground. In some embodiments, the kit includes instructions to remove 75% or more of the first fraction from the biological sample from the container. In other embodiments, the kit includes instructions to remove 75% or more of the first fraction from the biological sample from the container.

Compositions having a liquid exchanged biological fraction (e.g., a liquid exchanged platelet-rich fluid) are also provided. In some embodiments, the liquid exchanged platelet-rich fluid is produced from a blood sample (e.g., whole blood, anticoagulated whole blood) according to the methods described herein. The liquid exchanged platelet-rich fluid may include, in certain embodiments, little-to-no plasma. In some embodiments, the composition includes one or more electrolytes, proteins, activating compounds, ions as well as other active agents. In some embodiments, the subject compositions have a citrate ion concentration that is 10 mM or less, such as 5 mM or less, such as 1 mM or less. In certain instance, compositions having a liquid exchanged platelet-rich fluid do not have any citrate ions (i.e., a citrate ion concentration of 0 mM). In certain embodiments, the subject compositions have an albumin concentration that is 5 g/dl or less, such as 4.5 g/dl or less, such as 4 g/dl or less, such as 3.5 g/dl or less, such as 3 g/dl or less, such as 2.5 g/dl or less, such as 2 g/dl or less, such as 1.5 g/dl or less, such as 1 g/dl or less, such as 0.5 g/dl or less, such as 0.35 g/dl or less, such as 0.25 g/dl or less, such as 0.1 g/dl or less, such as 0.01 g/dl or less and including 0.001 g/dl or less. In certain embodiments, compositions having a liquid exchanged biological fraction do not have any albumin (i.e., an albumin concentration of 0 g/dl).

Aspects of the present disclosure also include methods for applying compositions having a liquid exchanged platelet-rich fluid to a body site of the subject. In some embodiments, the body site is a wound site (e.g., trauma or surgery), such as a wound site that is susceptible to forming an adhesion during wound repair. In some instances, the liquid exchanged platelet-rich fluid is prepared and applied directly to the wound site. In other instances, the liquid exchanged platelet-rich fluid is first prepared and supplemental active agents such as a drug, biological macromolecule (e.g., thrombin, cytokinin, fibrinogen) may be added to the composition before administering to the subject.

### BRIEF DESCRIPTION OF THE FIGURES

FIG. 1 shows a method of using a centrifuge container having a buoy in a prior art method of preparing biological fractions.

FIG. 2 shows a method of producing a liquid exchanged biological fraction according to an embodiment of the present disclosure.

### DETAILED DESCRIPTION

Methods of preparing liquid exchanged biological fractions are provided. Aspects of the methods according to certain embodiments include introducing a biological sample into a centrifugation container having a buoy, subjecting the biological sample to a force of centrifugation to produce two or more fractions in the biological sample, collecting a first fraction of the biological sample from the container and introducing a liquid exchange fluid into the container to produce a liquid exchanged fraction that is a mixture of the liquid exchange fluid and a second fraction of the biological sample. Compositions having a liquid exchanged platelet-rich fluid and kits for practicing embodiments of the subject methods are also provided. Methods for applying compositions having a liquid exchanged platelet-rich fluid to a body site of the subject are described.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods for Preparing a Liquid Exchanged Biological Fraction

As summarized above, aspects of the present disclosure include methods of preparing liquid exchanged biological fractions. By “liquid exchanged” biological fraction is meant a biological fraction that has had its initial liquid component, e.g., plasma, exchanged for another liquid component, such as a cell washing liquid or other liquid, e.g., as described in greater detail below. As such, a liquid exchanged biological fraction is a biological fraction in which the liquid components of the original source biological sample have been replaced by a second liquid (such as a synthetic, non-naturally occurring liquid) not part of the original source biological sample. In some instances, methods of using the buoy containing centrifugation separation devices to prepare washed cell concentrates are provided such that the cell washing effect is achieved by the substantial exchange of the first cell suspending fluid that the cells are suspended in when introduced into the device with a cell washing fluid before the cells are extracted from the device. The cell washing fluid substantially replaces the original cell suspending fluid of the extracted cells. Such cell washing is achieved, in some instances, with a single centrifugation step. The present disclosure provides for the preparation of a first cell composition from a heterogeneous cell containing biological fluid sample and the exchange of the first cell suspending fluid with a second suspending fluid (the cell wash fluid) with only a single centrifugation step. Previously it was necessary to perform one centrifugation step of the heterogeneous biological fluid to prepare the first cell composition and a second centrifugation step of the first cell composition to pellet the cells, to remove the first suspending fluid and then exchange it with a second suspending fluid to wash cells.

In embodiments, a multi-component liquid sample (e.g., a biological sample) is separated into fractions containing one or more of the components of the multi-component liquid sample. The term “separate” is used herein in its conventional sense to refer to the physical separation of a plurality of components based a particular physical or chemical property, such as density of the component. As described in greater detail below, the multi-component sample is introduced into a centrifugation container having a buoy configured to be displaced along a longitudinal axis within the container and subjected to a force of centrifugation for a duration sufficient to separate one or more components of the liquid sample. In embodiments, components are separated within the sample such that each component has an increased concentration in a particular region (e.g., distal end, proximal end or middle portion of the device container) as compared to the multi-component sample before centrifugation. In certain embodiments, the multi-component liquid sample is blood or a derivative thereof and methods includes separating components of the blood sample, such as separating white blood cells, red blood cells, plasma and platelets.

In embodiments, methods include separating components of the liquid sample into two or more regions (i.e., fractions) in the sample such that 5% or more of a certain component is separated into a particular region (e.g., distal end, proximal end or middle portion) of the device container, such as 10% or more, such as 20% or more, such as 25% or more, such as 30% or more, such as 40% or more, such as 50% or more, such as 60% or more, such as 70% or more, such as 80% or more, such as 90% or more, such as 95% or more and including separating 99% or more of a component into a particular region of the centrifugation container. In certain embodiments, 100% of the component is separated into a particular region of the centrifugation container. For example, where the multi-component liquid sample is a blood sample, methods include separating the blood sample in two or more fraction layers, such as three or more fraction layers in the centrifugation container. For example, in certain embodiments methods include centrifuging the container sufficient to form a layer of red blood cells at a distal end of the centrifugation container, a layer of platelet poor plasma at the proximal end of the centrifugation container and a layer of buffy coat on a surface of the buoy.

In embodiments, components of the sample may be separated into two or more fractions such that 5% or more of a certain component is separated in a particular fraction (e.g., bottom layer, upper layer, middle layer, etc.) of the sample, such as 10% or more, such as 20% or more, such as 25% or more, such as 30% or more, such as 40% or more, such as 50% or more, such as 60% or more, such as 70% or more, such as 80% or more, such as 90% or more, such as 95% or more and including separating 99% or more of a component into a particular fraction of the sample. In certain embodiments, 100% of the component is separated into a particular fraction of the sample.

In one example, the sample is whole blood or a derivative thereof (e.g., whole blood having one or more anticoagulants) and is subjected to a force of centrifugation for a duration sufficient to separate 90% or more of plasma into a first fraction, 90% or more of the buffy coat into a second fraction and 90% or more of red blood cells into a third fraction. For instance, the whole blood sample or derivative thereof is subjected to a force of centrifugation for a duration sufficient to separate 95% of the plasma into a first fraction, 95% of the buffy coat into a second fraction and 95% of the red blood cells into a third fraction.

In another example, the sample is bone marrow aspirate or a derivative thereof and is subjected to a force of centrifugation for a duration sufficient to separate 90% or more of a first component of the bone marrow aspirate into a first fraction, 90% or more of a second component of the bone marrow aspirate into a second fraction and 90% or more of a third component of the bone marrow aspirate into a third fraction. For instance, the bone marrow aspirate or derivative thereof is subjected to a force of centrifugation for a duration sufficient to separate 95% or more of a first component of the bone marrow aspirate into a first fraction, 95% or more of a second component of the bone marrow aspirate into a second fraction and 95% or more of a third component of the bone marrow aspirate into a third fraction.

As used herein, the term “multi-component liquid sample” is used to describe suspended media having more than one component, where multi-component liquid samples may include, but are not limited to, biological samples. The term “biological sample” is used in its conventional sense to include a whole organism, plant, fungi or a subset of animal tissues, cells or component parts which may in certain instances be found in blood (e.g., peripheral blood), mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs. Biological samples may include any type of organismic material, including both healthy and diseased components (e.g., cancerous, malignant, necrotic, etc.). Biological samples may include biologic fluids include bone marrow, phlegm, sputum, exudates, intestinal fluid, cell suspensions, tissue digests, tumor cell containing cell suspensions, microbe containing cell suspensions, and radiolabelled cell suspensions.

In certain embodiments, the biological sample is a liquid sample, such as whole blood or derivative thereof (e.g., plasma), tears, sweat, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.). The term “blood sample” refers to whole blood or a subset of blood components, including but not limited to platelets, red blood cells, white cells, buffy coat and blood plasma. The term “buffy coat” is used herein in its conventional sense to refer to the fractionated portion of blood of intermediate density (less dense than red blood cells, more dense than plasma) that contains white blood cells and platelets. In some embodiments, the blood sample is obtained from an in vivo source and can include blood samples obtained from tissues (e.g., bone marrow aspirate, cell suspension from a tissue biopsy, cell suspension from a tissue sample, etc.) or directly from a subject. In some cases, blood samples derived from a subject are cultured, stored, or manipulated prior to being subjected to separation.

In certain embodiments the source of the biological sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present disclosure may be applied to samples from a human subject, it is to be understood that the methods may also be carried-out on samples from other animal subjects (that is, in “non-human subjects”) such as, but not limited to, birds, mice, rats, dogs, cats, livestock and horses.

In practicing the subject methods, a biological sample is introduced into a centrifugation container having a buoy that is configured to be displaced along a longitudinal axis within the container. The multi-component liquid sample may be introduced into the container by any convenient liquid dispensing protocol, such as introducing the sample with a pipette, a syringe with or without a needle, a manual or mechanical dispenser or computer-automated liquid dispensing protocol. The volume of multi-component sample introduced into the container varies depending on the type of sample, size of device and amount of desired component recovery and ranges from 1 mL to 5000 mL, such as from 5 mL to 4500 mL, such as from 10 mL to 4000 mL, such as from 20 mL to 3500 mL, such as from 30 mL to 3000 mL, such as from 40 mL to 2500 mL, such as from 50 mL to 2000 mL, such as from 75 mL to 1500 mL and including from 100 mL to 1000 mL. In one example, methods include separating components of a 30 mL multi-component sample. In another example, methods include separating components of a 60 mL multi-component sample. In yet another example, methods include separating components of a 100 mL multi-component sample.

Centrifugation containers for separating components of a multi-component sample and for producing a liquid exchanged fraction as described herein include a buoy that is configured to be displaced along a longitudinal axis of the container. The term “displace” refers to movement of the buoy through the sample in the container during centrifugation. In embodiments, the buoy is displaced through the sample in response to the force of centrifugation. The buoy is displaced along the longitudinal axis within the container and can be displaced along all or part of the length of the inner cavity of the container during the subject methods, such as 25% or more of the length of the container, such as 35% or more, such as 50% or more, such as 60% or more, such as 75% or more, such as 90% or more, such as 95% or more, such as 97% or more and including 99% or more of the length of the container. In certain embodiments, the buoy is displaced along the entire (i.e., 100%) length of the container.

In response to centrifugation, the buoy is displaced proximally along the longitudinal axis of the container and takes a final position, depending on the type of multicomponent liquid sample that is on top of, below, or within the sample after centrifugation is complete. In some instances, the buoy takes a final position at the interface between a first separated fraction and a second separated fraction of the sample. In other instances, the buoy takes a final position within a fraction of the sample. In yet other instances, the buoy takes a final position that is on top of the fractionated sample. In still other instances, the buoy takes a final position that is below the fractionated sample (e.g., at the bottom of the container). For example, where the liquid sample is whole blood, methods include subjecting the whole blood sample to a force of centrifugation so that the buoy takes a final position at an interface between the fraction containing red blood cells and the fraction containing plasma. In other instances, the buoy takes a final position within the fraction containing red blood cells. In other instances, methods include subjecting the whole blood sample to a force of centrifugation so that the buoy takes a final position where the platelets, lymphocytes, monocytes and stem cells can be mixed with the liquid exchange fluid without substantial mixing with the granulocytes and red blood cells.

In embodiments, the biological sample is introduced into the centrifugation container, such as with a syringe through an inlet port. In some embodiments, the centrifugation container has a distal end and a proximal end with walls between the distal end and proximal end that together form an inner cavity within the container such that the buoy can freely be displaced along the longitudinal axis of the container during centrifugation without resistance by the walls of the container. In some embodiments, the outer walls of the container and inner cavity have the same cross-sectional shape where cross-sectional shapes of interest include, but are not limited to curvilinear cross-sectional shapes, e.g., circles, ovals, rectilinear cross sectional shapes, e.g., squares, rectangles, trapezoids, triangles, hexagons, etc., as well as irregular shapes, e.g., a parabolic bottom portion coupled to a planar top portion. For example, both the outer walls of the container and the inner cavity may have circular or oval cross sections or both the outer walls of the container and the inner cavity may have polygonal (e.g., octagonal) cross sections. In other embodiments, the outer walls and inner cavity of the container have different cross-sectional shapes (e.g., container having a polygonal cross-section and inner chamber having a circular cross-section). In certain embodiments, the container is a tube and the cross-sectional shape the outer walls and the inner walls are both circular.

Depending on the volume of the multi-component sample, the size of the inner cavity of the centrifugation container employed in the subject methods may vary, where in some instances the length of the inner cavity of the container may range from 1 cm to 25 cm, such as from 2.5 cm to 22.5 cm, such as from 5 cm to 20 cm, such as from 7.5 cm to 17.5 cm and including from 10 cm to 15 cm and the width of the inner cavity of the container may range from 1 cm to 20 cm, such as from 2 cm to 17.5 cm, such as from 3 cm to 15 cm, such as from 4 cm to 12.5 cm and including from 5 cm to 10 cm. Where the inner cavity of the container has a cylindrical cross-section, the diameter may vary, in some embodiments, ranging from 1 cm to 10 cm, such as from 2 cm to 9 cm, such as from 3 cm to 8 cm and including from 4 cm to 7 cm. Accordingly, the volume of the container may vary, ranging from 1 to 500 cm³, such as 5 to 250 cm³, such as 10 to 200 cm³, such as 15 to 150 cm³, such as 20 to 125 cm³ and including from 25 to 100 cm³. In some embodiments, the centrifugation container of is a tube having a volume ranging from 1 mL to 500 mL, such as from 2 mL to 400 mL, such as from 3 mL to 300 mL, such as from 4 mL to 200 mL, such as from 5 mL to 150 mL and including from 10 mL to 100 mL.

The multi-component sample may be introduced through one or more ports into the inner cavity of the centrifugation container. In certain embodiments, the centrifugation container includes a cap and the multi-component sample is introduced through a port in the cap. The ports may be any convenient port configured for fluidic or gaseous communication with the inner cavity of the container. In some embodiments, the cap includes a port for introducing the multi-component liquid sample into the container or a port for collecting components of the liquid after centrifugation (as described below). Where the cap includes a single port, the port is configured for both introducing the multi-component liquid sample into the container and for collecting one or more components from the cavity of the container after centrifugation.

Any suitable port configuration may be employed depending on the desired function of the port, where examples of ports include channels, orifices, channels having a check valve, a Luer taper fitting, a port with a breakable seal (e.g., single use ports) among other types of ports. In some embodiments, the port is configured to connect to a syringe, such as for example to introduce a multi-component liquid sample (e.g. blood) into the container or to remove one or more components from the container after centrifugation. In other embodiments, the port is configured to facilitate access for a needle into the cavity of the container to aspirate, mix and remove components from the container after centrifugation. In certain embodiments, the port is configured with a Luer taper fitting, such as a Luer-Lok or a Luer-slip.

In some embodiments, methods include releasably attaching a syringe to the one or more of the ports in the cap. For example, the cap may be configured to be non-permanently fastened to the syringe by a notch, a groove, a hook and loop fastener, Velcro, an adhesive, a threaded screw or a combination thereof. In some instances, the cap is configured to be releasably attached to the syringe by inserting the syringe into the orifice of the port. In other instances, the cap is configured to be screwed threaded with the syringe. In yet other instances, the cap is configured with a Luer taper fitting (e.g., Luer-Lok, Luer slip, etc.) and the syringe is releasably attached to the port in the cap through the Luer taper fitting.

In certain embodiments, the container also includes a conduit that extends from one or more of the ports in the cap to the proximal end of the buoy. In some embodiments, the port in the cap is in fluid communication with the proximal end of the buoy through the conduit. Put another way, the conduit includes two openings, a first opening in fluid communication with the port in the cap and a second opening in fluid communication with the proximal end of the buoy. The conduit may be integrated with or may be releasably attached to one or more of the port and the proximal end of the buoy. In some embodiments, the conduit is integrated with both the port in the cap and the buoy proximal end. The conduit may be integrated such as by co-molding, soldering, welding or affixing the conduit using a permanent adhesive. In other embodiments, the conduit is releasably attached to both the port in the cap and the proximal buoy end. The conduit may be releasably attached such as by non-permanently fastening with a notch, groove, snap-on, hook and loop fastener, Velcro, a threaded screw or with a non-permanent adhesive. In yet other embodiments, the conduit is integrated with the port in the cap and releasably attached to the proximal end of the buoy. In still other embodiments, the conduit is releasably attached to the port in the cap and integrated with the proximal end of the buoy. Depending on the size of the sample, the configuration of the conduit may vary. In some embodiments, the conduit is a linear tube extending from the port in the cap to the buoy proximal end. In other embodiments, the conduit is non-linear. For example, the conduit may be curvilinear, circular, winding, coiled, twisted or have a helical configuration.

In certain embodiments, the centrifugation container is a multi-component separation device having a buoy as described in United States Patent Publication No. 2017/0304823 as well as U.S. Pat. Nos. 8,632,736; 8,187,477; 7,837,884; 7,547,272; 7,470,371; 7,374,678; 7,077,273; 5,373,674, the disclosures of which are herein incorporated by reference.

In practicing the subject methods, the sample is subjected to a force of centrifugation one or more times. The term “force of centrifugation” is used herein in its conventional sense to refer to the force applied to the sample through revolving the device about an axis of rotation where the force on the components of the sample is in certain embodiments, given by the relative centrifugal force (RCF). The force of centrifugation may be applied by any convenient protocol, where in some embodiments, the force of centrifugation is applied by centrifuging the device with introduced sample with a centrifuge. In these embodiments, any convenient centrifuge may be employed, such as for example a fixed-angle centrifuge, a swinging bucket centrifuge, ultracentrifuge, solid bowl centrifuges, conical centrifuges, among other types of centrifuges. As described in greater detail below, the applied force of centrifugation (in relative centrifugal force, RCF) may vary depending on the sample type and size and may range from 1 g to 50,000 g, such as from 2 g to 45,000 g, such as from 3 g to 40,000 g, such as from 5 g to 35,000 g, such as from 10 g to 25,000 g, such as from 100 g to 20,000 g, such as from 500 g to 15,000 g and including from 1000 g to 10,000 g.

In some embodiments, the sample is subjected to the centrifugation force immediately after the sample is introduced into the subject separation centrifugation container. In other embodiments, the sample is subjected to the centrifugation force a predetermined period of time after introducing the sample into the centrifugation container. For example, the sample may be subjected to the centrifugation force 0.01 minutes or more after introducing the sample into the device container, such as after 0.05 minutes or more, such as after 0.1 minutes or more, such as after 0.5 minutes or more, such as after 1 minute or more, such as after 5 minutes or more, such as after 10 minutes or more, such as after 15 minutes or more, such as after 30 minutes or more and including 60 minutes after introducing the sample into the centrifugation container.

In certain embodiments, methods include a storage or prefabrication step where the sample is a specimen that has been preloaded into one or more of the subject separation devices and stored for a predetermined period of time before subjecting the sample to the centrifugation force. The amount of time the sample is preloaded and stored may vary, such as 0.1 hours or more, such as 0.5 hours or more, such as 1 hour or more, such as 2 hours or more, such as 4 hours or more, such as 8 hours or more, such as 16 hours or more, such as 24 hours or more, such as 48 hours or more, such as 72 hours or more, such as 96 hours or more, such as 120 hours or more, such as 144 hours or more, such as 168 hours or more and including preloading the sample for 240 hours or. For example, the amount of time the sample is preloaded and stored may range from 0.1 hours to 240 hours, such as from 0.5 hours to 216 hours, such as from 1 hour to 192 hours and including preloading the sample from 5 hours to 168 hours before subjecting the sample to the centrifugation force. For instance, the sample may be preloaded into one or more of the subject separation devices at a remote location (e.g., using in a physician's office or outpatient clinic) and sent to a laboratory for processing in accordance with the subject methods. By “remote location” is meant a location other than the location at which the sample is obtained and preloaded. For example, a remote location could be another location (e.g. office, lab, etc.) in the same city, another location in a different city, another location in a different state, another location in a different country, etc., relative to the location of the processing device, e.g., as described in greater detail below. In some instances, two locations are remote from one another if they are separated from each other by a distance of 10 m or more, such as 50 m or more, including 100 m or more, e.g., 500 m or more, 1000 m or more, 10,000 m or more, etc.

In embodiments, the sample is subjected to a force of centrifugation for a duration sufficient to separate components of different density into two or more fractions within the sample. The duration the sample is subjected to the force of centrifugation may vary and may be 0.01 minutes or longer, such as for 0.05 minutes or longer, such as for 0.1 minutes or longer, such as for 0.5 minutes or longer, such as for 1 minute or longer, such as for 3 minutes or longer, such as for 5 minutes or longer, such as for 10 minutes or longer, such as for 15 minutes or longer, such as for 20 minutes or longer, such as for 30 minutes or longer, such as for 45 minutes or longer, such as for 60 minutes or longer and including for 90 minutes or longer. For example, the sample may be subjected to force of centrifugation for a duration which ranges from 0.01 minutes to 960 minutes, such as from 0.05 minutes to 480 minutes, such as from 0.1 minutes to 240 minutes, such as from 0.5 minutes to 120 minutes, such as from 1 minute to 90 minutes, such as from 5 minutes to 60 minutes and including from 10 minutes to 45 minutes.

Depending on the volume of sample and density dispersity of the sample components, the rotational speed of centrifugation may vary, such as from 1×10³ revolutions per minute (rpm) to 1000×10³ rpm, such as from 2×10³ rpm to 900×10³ rpm, such as from 3×10³ rpm to 800×10³ rpm, such as from 4×10³ rpm to 700×10³ rpm, such as from 5×10³ rpm to 600×10³ rpm, such as from 10×10³ rpm to 500×10³ rpm and including from 25×10³ rpm to 100×10³ rpm. The centrifuge may be maintained at a single speed or may be changed to a different speed at any time during separation of the sample components. Where the centrifuge is operated at more than one speed, the duration the centrifuge is maintained at each speed may independently be 0.01 minutes or more, such as 0.1 minutes or more, such as 1 minute or more, such as 5 minutes or more, such as 10 minutes or more, such as 30 minutes or more and including 60 minutes or more. The time period between each different speed employed may also vary, as desired, being separated independently by a delay of 1 minute or more, such as 5 minutes or more, such as by 10 minutes or more, such as by 15 minutes or more, such as by 30 minutes or more and including by 60 minutes or more. In embodiments where the centrifuge is maintained at more than two (i.e., three or more) speed to subject the sample to the centrifugation force, the delay between each speed employed may be the same or different.

Depending on the type and number of components of different density in the sample the centrifuge may be maintained at a speed to subject the sample to the centrifugation force continuously or in discrete intervals. For example, in some embodiments, the centrifuge is maintained at a speed to subject the sample to a centrifugation force continuously. In other instances, the centrifuge is maintained at a speed to subject the sample to a centrifugation force in discrete intervals, such as for example for intervals of for 0.01 minutes or longer, such as for 0.05 minutes or longer, such as for 0.1 minutes or longer, such as for 0.5 minutes or longer, such as for 1 minute or longer, such as for 3 minutes or longer, such as for 5 minutes or longer, such as for 10 minutes or longer, such as for 15 minutes or longer, such as for 20 minutes or longer, such as for 30 minutes or longer, such as for 45 minutes or longer, such as for 60 minutes or longer and including for 90 minutes or longer. Where the centrifuge is maintained at a speed in discrete intervals, methods may include 1 or more intervals, such as 2 or more intervals, such as 3 or more intervals and including 5 or more intervals.

The rotational speed of centrifugation during each step may vary, as described above, such as where the speed of centrifugation ranges from 1×10³ revolutions per minute (rpm) to 1000×10³ rpm (e.g., from 2×10³ rpm to 500×10³ rpm) and the speed of centrifugation when the centrifuge activated valve is in the closed position ranges from 1×10³ revolutions per minute (rpm) to 1000×10³ rpm (e.g., from 2×10³ rpm to 500×10³ rpm).

In some embodiments, the sample is subjected to a single interval of centrifugation. By “single interval” is meant that a force of centrifugation is applied only once to the sample in the centrifugation container to separate fractions of the multi-component sample as desired. In other words, the desired separation of components of the sample is achieved by a single application of the force of centrifugation to the sample and the liquid exchange fluid is mixed with the one or more fractions after a single interval of centrifugation.

In embodiments of the present disclosure, each step (introduction of the sample into the separation device container, subjecting the sample to a centrifugation force, introducing the liquid exchange fluid into the container to produce a liquid exchanged fraction and collecting the liquid exchanged fraction from the container) can be carried out at any suitable temperature so long as the viability of the components (e.g., platelets) of the sample are preserved as desired. As such, the temperature according to embodiments of the disclosure may vary, such as from −80° C. to 100° C., such as from −75° C. to 75° C., such as from −50° C. to 50° C., such as from −25° C. to 25° C., such as from −10° C. to 10° C., and including from 0° C. to 25° C.

Where necessary, the parameters for subjecting the samples to a centrifugation force may be changed at any time during methods of the present disclosure. For example, the speed of the centrifuge, the duration the sample is subjected to the centrifugation force and heating or cooling of the sample may be changed one or more times during the subject methods, such as two or more times, such as three or more times and including five or more times.

In some embodiments, methods include changing the speed of the centrifuge, such as by increasing or decreasing the speed by 1% or more, such as by 5% or more, such as by 10% or more, such as by 25% or more, such as by 50% or more, such as by 75% or more, such as by 90% or more, such as by 2-fold or more, such as by 5-fold or more, such as by 10-fold or more and including by 25-fold or more. For example, the speed of the centrifuge may be increased or decreased by 0.5×10³ rpm or more, such as by 1×10³ rpm or more, such as by 2×10³ rpm or more, such as by 5×10³ rpm or more, such as by 10×10³ rpm or more, such as by 25×10³ rpm or more and including increasing or decreasing the speed of the centrifuge by 100×10³ rpm or more.

In other embodiments, the duration the sample is subjected to the centrifugation force may be changed. For example, the duration the sample is subjected to the centrifugation force may be increased or decrease by 0.01 minutes or longer, such as by 0.05 minutes or longer, such as by 0.1 minutes or longer, such as by 0.5 minutes or longer, such as by 1 minute or longer, such as by 3 minutes or longer, such as by 5 minutes or longer, such as by 10 minutes or longer, such as by 15 minutes or longer, such as by 20 minutes or longer, such as by 30 minutes or longer, such as by 45 minutes or longer, such as by 60 minutes or longer and including by 90 minutes or longer.

In yet other embodiments, the temperature while subjecting the sample to the centrifugation force may be changed. For example, the temperature may be raised or lower by 0.1° C. or more, such as by 0.5° C. or more, such as by 1° C. or more, such as by 2° C. or more, such as by 5° C. or more and including raising or lowering the temperature by 8° C. or more.

In certain embodiments, methods include monitoring the centrifuged sample. Monitoring may include assessing (either by a human or with the assistance of a computer, if using a computer-automated process initially set up under human direction) the extent of component separation within the sample. For example, monitoring separation of components by density into the two or more fractions within the sample may include visually determining fraction boundaries between components of the sample. Monitoring separation of components may also include assessing the physical and chemical properties of the components in each fraction within the sample. Any convenient protocol can be employed to monitor the sample, including but not limited to visual observation, laser scatter, fluorescence, phosphorescence, chemiluminescence, diffuse reflectance, infrared spectroscopy, among other sensing protocols.

In some instances, monitoring includes collecting real-time data, such as employing a detector (e.g., with a video camera). In other instances, monitoring includes assessing the sample at regular intervals, such as every 0.01 minutes, every 0.05 minutes, every 0.1 minutes, every 0.5 minutes, every 1 minute, every 5 minutes, every 10 minutes, every 30 minutes, every 60 minutes or some other interval.

Methods of the present disclosure may also include a step of assessing the sample to identify any desired adjustments to the subject protocol. In other words, methods in these embodiments include providing feedback based on monitoring the sample, where adjustments to the protocol may vary in terms of goal, where in some instances the desired adjustment are adjustments that ultimately result in an improved fractionation of components by density within the sample, such as providing faster separation, improved purity and content of the produced liquid exchanged fraction.

Where feedback provided indicates that a particular protocol is less than optimal, methods may include changing one or more parts of the subject protocols. For example, one or more parameters for subjecting the sample to a centrifugation force may be adjusted. In one example, methods include adjusting the speed of the centrifuge. In another example, methods include changing (increasing or decreasing) the duration the sample is subjected to the centrifugation force. In yet another example, methods include heating or cooling the sample.

In practicing the subject methods, one or more of the separated fractions may be collected. Fractions may be collected using any suitable collecting protocol, such as aspirating using a syringe with or without a needle, a manual or mechanically operated serological pipette as well as with an automated liquid collection system (e.g., a computer-controlled collection apparatus). Where the sample is portioned into two or more of the subject devices, collecting fractions may include combining fractions of similar makeup. For example, where a whole blood or bone marrow aspirate sample is fractionated using two or more of the subject devices, buffy coat from each of the devices may be collected (e.g., from the surface of a centrifuge activated suspension floor of the centrifuge activated valve on the buoy) and combined.

Separated fractions may be collected from the sample at any time after subjecting the sample to the force of centrifugation. In some embodiments, the separated fractions are collected 1 minute or greater after the separated fractions are prepared, such as 2 minutes or greater, such as 3 minutes or greater, such as 5 minutes or greater, such as 10 minutes or greater and including 30 minutes or greater after the separated fractions are prepared.

All or part of the fraction may be removed from the centrifuge container, such as 15% or more of the fraction, such as 25% or more, such as 50% or more, such as 75% or more and including removing 95% or more of the fraction. In certain instances, the entire fraction (i.e., 100%) is removed from the centrifuge container. In some embodiments, the sample is a whole blood sample and methods include removing all or part of a platelet-poor plasma fraction. For example, 15% or more of the platelet-poor plasma fraction may be removed, such as 25% or more, such as 50% or more, such as 75% or more and including removing 95% or more of the platelet-poor plasma fraction. In other embodiments, methods include removing all or part of a red blood cell fraction from the centrifuge container, such as 15% or more of the red blood cell fraction, such as 25% or more, such as 50% or more, such as 75% or more and including removing 95% or more of the red blood cell fraction.

To remove the fraction from the container, methods may include positioning a liquid collection device (e.g., needle with syringe) a predetermined depth into the fraction. For example, the liquid collection device may be positioned 1 mm or more into fraction, such as 2 mm or more, such as 3 mm or more, such as 5 mm or more, such as 10 mm or more and including 25 mm or more into the fraction. In certain embodiments, the liquid collection device is positioned to a depth as determined by one or more reference indicators on the container. In still other embodiments, the liquid collection device is positioned to a depth relative to the outer edge of the buoy proximal end, such as 1 mm or more above the outer edge of the buoy proximal end, such as 2 mm or more, such as 3 mm or more, such as 5 mm or more, such as 10 mm or more and including 25 mm or more above the outer edge of the buoy proximal end. In certain instances, methods for removing a portion of the fraction include positioning the liquid collection device directly against the outer edge of the buoy proximal end.

In some embodiments, to remove the fraction from the container, the container is positioned at an angle with respect to an axis orthogonal to the ground. For example, the fraction may be removed by tilting the container to an angle that is be 5° or more with respect to an axis orthogonal to the ground, such as 10° or more, such as 15° or more, such as 25° or more, such as 30° or more, such as 45° or more, such as 60° or more, such as 75° or more and including 80° or more. For example, the device may be tilted to an angular position that ranges from 1° to 90° with respect to an axis orthogonal to the ground, such as from 5° to 85°, such as from 10° to 80°, such as from 15° to 75°, such as from 20° to 70° and including from 3° to 60°.

In embodiments, after a first fraction of the biological sample is removed from the centrifuge container, a liquid exchange fluid is introduced into the container to produce a liquid exchanged fraction that is a mixture of the liquid exchange fluid and a second fraction of the biological sample. For example, where the biological sample is a blood sample, liquid exchange fluid may be introduced into the container after removing a platelet-poor plasma fraction and mixing the liquid exchange fluid with a buffy coat and white blood cell fraction to produce a liquid exchanged platelet-rich buffy coat and white blood cell fraction.

The liquid exchange fluid may be any suitable fluid that is miscible with the separated fraction from the biological sample, where, in certain embodiments, the liquid exchange fluid is an aqueous composition. In some instances, the liquid exchange fluid includes one or more of electrolytes, proteins, activating compounds, ions as well as other active agents. In some embodiments, the liquid exchange fluid includes a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof. In certain embodiments, the liquid exchange fluid is a buffered saline. The liquid exchange fluid in some instances has a citrate ion concentration that is 10 mM or less, such as 5 mM or less, such as 1 mM or less. In certain instance, the liquid exchange fluid does not have any citrate ions (i.e., a citrate ion concentration of 0 mM).

In some embodiments, after removing the first fraction, the container is tilted to a second angular position with respect to the axis orthogonal to the ground and the liquid exchange fluid is introduced into the container mixed with the second fraction (e.g., in the buoy). The container may be tilted to any suitable angular position, depending on the position of the port in the cap of the container and the volume of the biological sample and may be 5° or more with respect to an axis orthogonal to the ground, such as 10° or more, such as 15° or more, such as 25° or more, such as 30° or more, such as 45° or more, such as 60° or more, such as 75° or more and including 80° or more. For example, the device may be tilted to a second angular position that ranges from 1° to 90° with respect to an axis orthogonal to the ground, such as from 5° to 85°, such as from 10° to 80°, such as from 15° to 75°, such as from 20° to 70° and including from 3° to 60°. In other embodiments, the device is tilted to a second angular position that is 5° or more with respect to the first angular position, such as 10° or more, such as 15° or more, such as 25° or more, such as 30° or more, such as 45° or more, such as 60° or more, such as 75° or more and including 80° or more. For example, the device may be tilted to a second angular position that 1° to 90° with respect to the first angular position, such as from 5° to 85°, such as from 10° to 80°, such as from 15° to 75°, such as from 20° to 70° and including from 3° to 60°.

In some embodiments, where the first fraction is removed from the container by tilting the container to an angle with respect to an axis orthogonal to the ground, methods include maintaining the centrifuge container at the tilted angle and introducing the liquid exchange fluid into the container to produce the liquid exchanged fraction. In these embodiments, the container is maintained at the angle that the first fraction is removed, such as maintaining the container at an angle that is 5° or more with respect to an axis orthogonal to the ground, such as 10° or more, such as 15° or more, such as 25° or more, such as 30° or more, such as 45° or more, such as 60° or more, such as 75° or more and including 80° or more. For example, the device may maintained at the angle of from 1° to 90° with respect to an axis orthogonal to the ground to introduce and mix the liquid exchange fluid with the second fraction, such as from 5° to 85°, such as from 10° to 80°, such as from 15° to 75°, such as from 20° to 70° and including from 3° to 60°.

The liquid exchange fluid may be introduced into the centrifugation device by any convenient liquid dispensing protocol, such as introducing the sample with a pipette, a syringe with or without a needle, a manual or mechanical dispenser or computer-automated liquid dispensing protocol. The volume of liquid exchange fluid introduced into the container varies depending on the type of sample, size of centrifuge container and amount of desired liquid exchange fraction and ranges from 1 mL to 5000 mL, such as from 5 mL to 4500 mL, such as from 10 mL to 4000 mL, such as from 20 mL to 3500 mL, such as from 30 mL to 3000 mL, such as from 40 mL to 2500 mL, such as from 50 mL to 2000 mL, such as from 75 mL to 1500 mL and including from 100 mL to 1000 mL.

Mixing the liquid exchange fluid with the second fraction may be repeated as desired, such as 2 or more times, such as 3 or more times, such as 4 or more times, such as 5 or more times and including 10 or more times. After the liquid exchange fluid and the second fraction are sufficiently mixed as desired (e.g., adequately mixed platelet-rich liquid exchange fraction is produced), the mixture may be collected by any convenient liquid collection protocol, such as with a syringe with or without a needle, a manual or mechanically operated serological pipette as well as with an automated liquid collection system (e.g., a computer-controlled collection apparatus).

In some embodiments as described above, positioning the container at an angle includes placing the container into an adjustable tilter stand and adjusting the container to the desired angle (e.g., an angle ranging from 10 degrees to 60 degrees relative to an axis orthogonal to the ground). For example, the container may be placed into the adjustable stand and the container may be adjusted to the desired angle while visually monitoring the position of the conduit opening with respect to the first fraction. The stand may be any suitable support protocol, including by not limited to a manual support or with a manual, mechanical or automated actuator. In some embodiments, the stand includes a platform with a hinge or latch at a proximal edge of the container where the actuator positions a support at an angle by raising the distal edge of the container. In other embodiments, the stand protocol is a tilter stand configured to receive the device into a receptacle and to position the device at any desired angular position. In other embodiments, the actuator may be a lift column that is coupled to the bottom of the container and positioning of the container by the actuator at an angle includes adjusting a pivot or rocker. In some instances, actuation of the container to the desired angle is carried out manually (i.e., positioning of the container by hand).

In certain embodiments, the container is positioned at an angle by placing the container into an adjustable tilter stand as described in United States Patent Publication No. 2017/0304823, the disclosure of which is herein incorporated by reference.

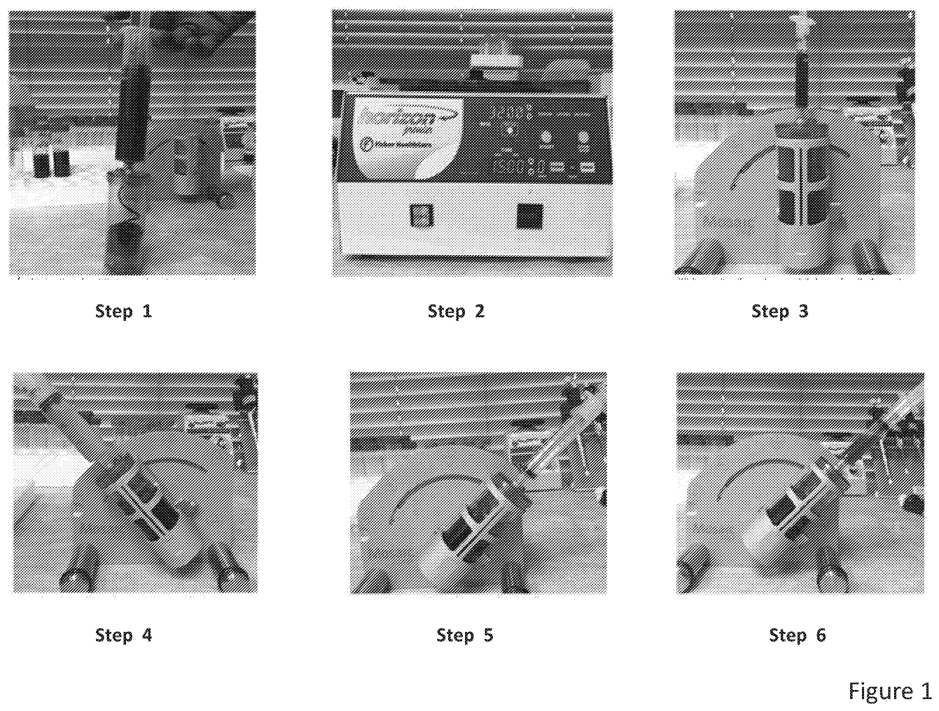

FIG. 1 depicts using a centrifuge container having a buoy in a prior art method for preparing fractions from a multi-component biological sample. At step 1, 2 mL of a multi-component biological sample (anticoagulated whole blood) is introduced into the centrifuge container having a buoy. At step 2, the sample loaded container is centrifuged (e.g., for 15 minutes at 3200 RPM). At step 3, the container with centrifuged sample is placed onto a tilter stand and 2 mL of the red blood cell fraction is removed from the container with a 3 mL syringe. At step 4, the container is tilted to a first angle (e.g., −30°) and with a 30 mL syringe, the plasma component (i.e., platelet-poor plasma) is removed from the container. At step 5, the container is tilted to a second angle (e.g., 30°) and with a 10 mL syringe, the platelet rich plasma fraction is withdrawn into the syringe and re-injected at a rapid rate. At step 6, after repeating the withdrawing and re-injection of the platelet rich plasma fraction, the platelet rich plasma of the biological sample is removed from the container.

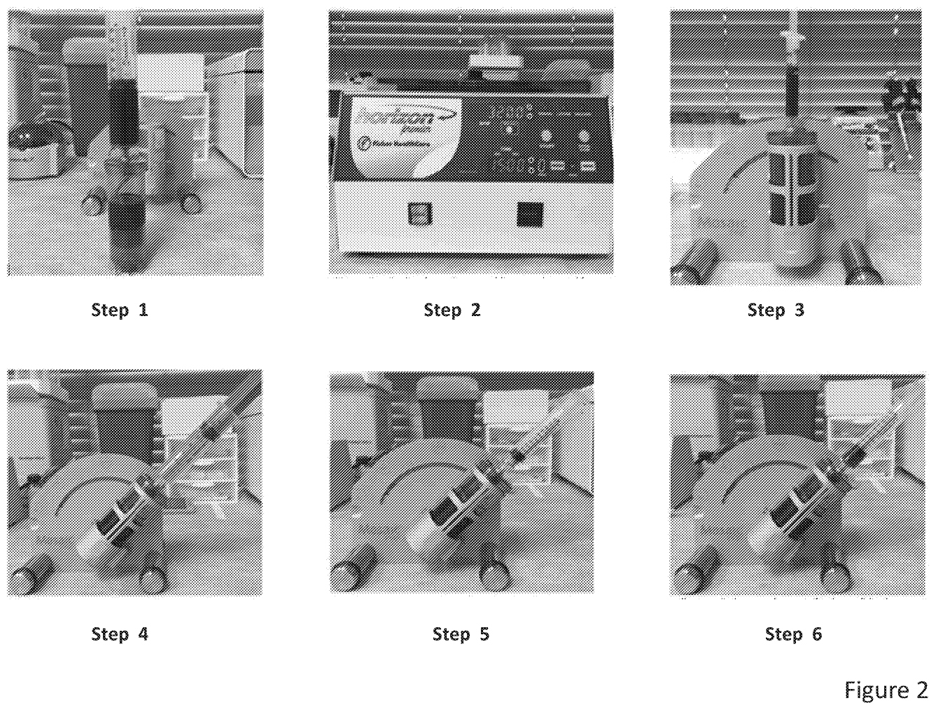

FIG. 2 depicts using a centrifuge container for producing a liquid exchanged biological fraction according to certain embodiments of the present disclosure. At step 1, 2 mL of a multi-component biological sample (anticoagulated whole blood) is introduced into the centrifuge container having a buoy. At step 2, the sample loaded container is centrifuged (e.g., for 15 minutes at 3200 RPM). At step 3, the container with centrifuged sample is placed onto a tilter stand and 2 mL of the red blood cell fraction is removed from the container with a 3 mL syringe. At step 4, the container is tilted to a first angle (e.g., 30°) and with a 30 mL syringe, the plasma component (i.e., platelet-poor plasma) is removed from the container. At step 5, the container is maintained at the first angle and with a 10 mL syringe a liquid exchange fluid (e.g., saline) is injected into the container at a rapid rate. The liquid exchange fluid mixes with a second fraction (e.g., white blood cells and buffy coat) and forms a liquid exchanged fraction in the centrifuge container. This liquid exchanged fraction is withdrawn and re-injected into the container for additional mixing. At step 6, after repeating the withdrawing and re-injection of the liquid exchanged fraction, the liquid exchanged platelet-rich saline is removed from the container.

### Compositions Having a Liquid Exchanged Biological Fraction

As summarized above, aspects of the present disclosure include a composition having a liquid exchanged biological fraction. As described above, the term “liquid exchanged” refers to a biological fraction that has had its initial liquid component, e.g., plasma, exchanged for another liquid component, such as a cell washing liquid or other liquid. As such, a liquid exchanged biological fraction is a biological fraction in which the liquid components of the original source biological sample have been replaced by a second liquid (such as a synthetic, non-naturally occurring liquid) not part of the original source biological sample. In some embodiments, the biological sample is a blood sample (e.g., whole blood, anticoagulated whole blood) and the composition includes a liquid exchanged platelet-rich fluid (e.g., a liquid exchanged platelet-rich buffered saline). In certain instances, the liquid exchanged platelet-rich fluid includes little if any plasma (and in some instances includes no plasma), such as where the plasma content (e.g., the plasma from the original blood sample) is present in the liquid exchanged platelet-rich fluid in an amount of 10% by volume or less, such as 9% by volume or less, such as 8% by volume or less, such as 7% by volume or less, such as 6% by volume or less, such as 5% by volume or less, such as 4% by volume or less, such as 3% by volume or less, such as 2% by volume or less, such as 1% by volume or less, such as 0.5% by volume or less, such as 0.1% by volume or less, such as 0.01% by volume or less and including 0.001% by volume or less. In certain embodiments, the biological sample is a stem cell containing biological sample and compositions of interest include a liquid exchanged stem cell-rich fluid.

In certain embodiments, the subject composition is an aqueous fluid. In some embodiments, the liquid exchange fluid is a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof. In some instances, compositions of interest includes one or more electrolytes, proteins, activating compounds, ions as well as other active agents. In certain embodiments, composition has a citrate ion concentration that is 10 mM or less, such as 5 mM or less, such as 1 mM or less. In certain instance, the composition does not have any citrate ions (i.e., a citrate ion concentration of 0 mM). In certain embodiments, the subject compositions have an albumin concentration that is 5 g/dl or less, such as 4.5 g/dl or less, such as 4 g/dl or less, such as 3.5 g/dl or less, such as 3 g/dl or less, such as 2.5 g/dl or less, such as 2 g/dl or less, such as 1.5 g/dl or less, such as 1 g/dl or less, such as 0.5 g/dl or less, such as 0.35 g/dl or less, such as 0.25 g/dl or less, such as 0.1 g/dl or less, such as 0.01 g/dl or less and including 0.001 g/dl or less. In certain embodiments, compositions having a liquid exchanged biological fraction do not have any albumin (i.e., an albumin concentration of 0 g/dl).

In some embodiments, the composition includes viable stem cells. The term “stem cells” is used herein in its conventional sense to refer to undifferentiated biological cells that can differentiate into specialized cells and can divide to produce more stem cells, such as for example mesenchymal stem cells and stromal stem cells. By “viable” is meant that the stem cells are living and capable of maintaining or recovering the potentialities of stem cell activity (e.g., dividing, differentiating, etc.)

In some embodiments, the composition includes tissue plasminogen activator. Tissue plasminogen activator refers to the serine protease protein that catalyzes the conversion of plasminogen to plasmin. The amount of tissue plasminogen activator in the subject compositions may vary, ranging from 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of the tissue plasminogen activator may range from 1×10² IU/mg to 1×10⁸ IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

In other embodiments, the composition includes thrombin. The amount of thrombin may vary and may include 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of the thrombin may range from 1×10² to 1×10⁸ IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

In still other embodiments, the compositions include plasminogen. The amount of plasminogen in the subject compositions may vary, ranging from 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of plasminogen may range from 1×10² IU/mg to 1×10⁸IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

In still other embodiments, the compositions include fibrinogen. The amount of fibrinogen in the subject compositions may vary, ranging from 0.01 μg to 100 μg, such as from 0.05 μg to 90 μg, such as from 0.1 μg to 80 μg, such as from 0.5 μg to 70 μg, such as from 1 μg to 60 μg and including from 5 μg to 50 μg. In these embodiments, the expressed activity of fibrinogen may range from 1×10² IU/mg to 1×10⁸ IU/mg, such as from 5×10² IU/mg to 5×10⁷ IU/mg, such as from 1×10³ IU/mg to 1×10⁷ IU/mg, such as from 5×10³ IU/mg to 5×10⁶ IU/mg and including from 1×10⁴ IU/mg to 1×10⁶ IU/mg.

In some embodiments, the compositions of interest include one or more bioactive agents, such as for delivering the one or more bioactive agent to a site of administration, as described in greater detail below. The subject compositions having a liquid exchanged biological fraction may include one or more types of bioactive agents, such as two or more types, such as three or more types, such as four or more types, such as five or more types and including ten or more types of bioactive agents.

Example bioactive agents according to embodiments of the disclosure may include but are not limited to interferon, interleukin, erythropoietin, granulocyte-colony stimulating factor (GCSF), stem cell factor (SCI:), leptin (OB protein), interferon (alpha, beta, gamma), antibiotics such as vancomycin, gentamicin ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, glial derived neurotrophic factor (GDNF), neurotrophic factor 3 (NT3), fibroblast growth factor (FGF), transforming growth factor (TGF), platelet transforming growth factor, milk growth factor, endothelial growth factors, endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-IBBR), TRAIL (TNF-related apoptosis inducing ligand), artemin (GFRalpha3-RET ligand), BCA-I (B cell-attracting chemokinel), B lymphocyte chemoattractant (BLC), B cell maturation protein (BCMA), brain-derived neurotrophic factor (BDNF), bone growth factor such as osteoprotegerin (OPG), bone-derived growth factor, thrombopoietin, megakaryocyte derived growth factor (MDGF), keratinocyte growth factor (KGF), platelet-derived growth factor (PDGF), ciliary neurotrophic factor (CNTF), neurotrophin 4 (NT4), granulocyte colony-stimulating factor (GCSF), macrophage colony-stimulating factor (mCSF), bone morphogenetic protein 2 (BMP2), BRAK, C-IO, Cardiotrophin 1 (CTI), CCR8, anti-inflammatory: paracetamol, salsalate, diflunisal, mefenamic acid, diclofenac, piroxicam, ketoprofen, dipyrone, acetylsalicylic acid, anti-cancer drugs such as aliteretinoin, altertamine, anastrozole, azathioprine, bicalutarnide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, antivirus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetaminophen, analgesics, antitumor preparations, anti-cancer preparations, anti-proliferative preparations or pro-apoptotic preparations, among other types of bioactive agents.

The amount of each bioactive agent in the subject compositions having a liquid exchange biological fraction may vary depending on the type of bioactive agent and may be 0.01 μg or more, such as 0.05 μg or more, such as 0.1 μg or more, such as 0.5 μg or more, such as 1 μg or more, such as 5 μg or more, such as 10 μg or more, such as 25 μg or more, such as 50 μg or more, such as 100 μg or more, such as 250 μg or more, such as 1000 μg or more, such as 10 g or more, such as 25 g or more and including 100 g of bioactive agent or more. For example, the concentration of each bioactive agent may be 0.0001 μg/mL or greater, such as 0.001 μg/mL or greater, such as 0.01 μg/mL or greater, such as 0.1 μg/mL or greater, such as 0.5 μg/mL or greater, such as 1 μg/mL or greater, such as 2 μg/mL or greater, such as 5 μg/mL or greater, such as 10 μg/mL or greater, such as 25 μg/mL or greater, such as 50 μg/mL or greater, such as 100 μg/mL or greater such as 500 μg/mL or greater, such as 1 g/mL or greater such as 5 g/mL or greater and including 10 g/mL or greater.

Where the composition includes more than one bioactive agent, the amount (i.e., mass) of each of bioactive agent may vary, ranging from 0.001 mg to 1000 mg, such as 0.01 mg to 500 mg, such as 0.1 mg to 250 mg, such as 0.5 mg to 100 mg, such as 1 mg to 50 mg, including 1 mg to 10 mg. As such, in the subject compositions, the mass ratio of the first bioactive agent to other (i.e., second or more) bioactive agent may vary, and in some instances may range between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. For example, the mass ratio of the first bioactive agent to other (i.e., second or more) bioactive agents may range between 1:1 and 1:10; 1:5 and 1:25; 1:10 and 1:50; 1:25 and 1:100; 1:50 and 1:500; or 1:100 and 1:1000.

### Methods for Applying a Composition Having a Liquid Exchanged Biological Fraction to a Subject

Aspects of the disclosure also include methods for applying one or more of the compositions having a liquid exchanged biological fraction (e.g., a composition having a liquid exchanged platelet-rich fluid) to a subject. Methods of using the composition include administering one or more of the compositions to a body site of the subject in order to treat a subject for a target condition of interest such as wound repair (in trauma wounds or surgical wounds). By “treating” or “treatment” is meant at least a suppression or amelioration of the symptoms associated with the condition affecting the subject, where suppression and amelioration are used in a broad sense to refer to at least a reduction in the magnitude of a parameter, e.g., symptom, associated with the condition being treated. As such, treatment also includes situations where the condition is completely inhibited, e.g., prevented from happening, or stopped, e.g., terminated, such that the subject no longer experiences the condition. As such, treatment includes both preventing and managing a condition.

In certain embodiments, the subject compositions having a liquid exchanged biological fraction as described above are used in the treatment of a wound site, such as where the wound occurs by injury or by surgery. In other embodiments, the subject compositions are used to facilitate healing of connective tissue. In still other embodiments, the subject compositions are used to reduce time for wound closure. In still other embodiments, the subject compositions are used to deliver one or more bioactive agents to the body site, such as by sustained or pulsatile release.

In embodiments, the methods include applying one or more of the subject compositions to a body site of a subject, such as a wound site. The term “subject” is meant the person or organism to which the composition having the liquid exchanged biological fraction is applied. As such, subjects may include but are not limited to mammals, e.g., humans and other primates, such as chimpanzees and other apes and monkey species; and the like, as well as non-human subjects such as, but not limited to, birds, mice, rats, dogs, cats, livestock and horses. In certain embodiments, the subject is a human.

In practicing the subject methods, the composition may be applied to any convenient internal or external location on the subject, such as to organ tissue including but not limited to integumentary tissue (e.g. sections of the skin), oral tissue (e.g., buccal, tongue, palatal, gums), respiratory tissue (e.g., pharynx, larynx, trachea, bronchi, lungs, diaphragm) gastrointestinal tissue (e.g., esophagus, stomach, liver, gallbladder, pancreas, intestines, colon, rectum and anus.), cardiovascular tissue (e.g., heart, blood vessels), endocrine tissue (e.g., hypothalamus, pituitary gland, pineal body or pineal gland, thyroid, parathyroids, adrenal glands) and genitourinary tissue (kidneys, ureters, bladder, urethra, ovaries, fallopian tubes, uterus, vagina, mammary glands, testes, vas deferens, seminal vesicles, prostate, penis), muscular tissue, nervous tissue (e.g., brain, spinal cord, nerves) as well as soft skeletal tissue (cartilage, ligaments, tendons). Furthermore, the composition may be applied to any type of organismic tissue, including both healthy and diseased tissue (e.g., cancerous, malignant, necrotic, etc.), where desired.

The size of the body site (e.g., wound site) treated with the subject compositions may vary, such as having a surface area ranging from 0.1 to 100 cm², such as 0.5 to 75 cm², such as 1.0 to 50 cm², such as 1.5 to 45 cm², such as 2.0 to 40 cm², such as 2.5 to 35 cm², and including 2 to 30 cm². Where body site treated is three-dimensional cavity, the size of the body site may range from 0.1 to 100 cm³, such as 0.5 to 75 cm³, such as 1.0 to 50 cm³, such as 1.5 to 45 cm³, such as 2.0 to 40 cm³, such as 2.5 to 35 cm³, and including 2 to 30 cm³. The subject compositions may be applied and maintained at the application site over an extended period of time, as desired. For example, the composition having a liquid exchanged biological fraction (e.g., a liquid exchanged platelet-rich fluid) may be maintained at the body site (e.g., wound site) over the course of hours, days and including weeks.

In practicing the subject methods, the composition may be applied a single time or a plurality of times over a given time period, e.g., during the course of wound repair, where the application schedule when a plurality of composition are applied over a given time period may be hourly, daily, weekly, etc. For example, the subject methods include multiple application intervals. By “multiple application intervals” is meant more than one composition is applied to the subject in a sequential manner. In practicing the subject methods, treatment regimens may include two or more application intervals, such as three or more application intervals, such as four or more application intervals, such as five or more application intervals, including ten or more application intervals.

The duration between application intervals in a multiple application interval treatment regimen may vary, as determined by a qualified health care professional. For example, the duration between application intervals in a multiple application treatment regimen may be predetermined and follow at regular intervals. As such, the time between application intervals may vary and may be 1 hour or longer, such as 2 hours or longer, such as 3 hours or longer, such as 6 hours or longer, such as 12 hours or longer, such as 24 hours or longer, such as 48 hours or longer, such as 72 hours or longer, including 168 hours or longer.

In certain embodiments, the subject methods include assessing a subject as in need of treatment with one or more of the subject compositions described above. Individuals may be assessed using any convenient protocol. For example, methods may include determining that a subject has a wound that can be treated by the subject composition, such as post-surgical wound or a connective tissue injury. Diagnosis or assessment of target condition can be performed using any convenient diagnostic protocol as determined by a qualified health care professional.

In certain embodiments, the subject compositions having a liquid exchanged biological fraction (e.g., a liquid exchanged platelet-rich fluid) can be applied concurrent with other therapeutic protocols, such as for example, for management of blood clotting (e.g., anticoagulation protocols, anti-thrombotic protocols), as well as with devices to physically separate tissues at the body site (e.g., to inhibit adhesion formation). By “concurrent application” is intended administration to a subject such that the therapeutic effect of the combination is caused in the subject undergoing therapy.

In certain embodiments, methods include delivering one or more bioactive agents to the body site with the subject compositions. In these embodiments, methods may include delivering one or more types of bioactive agents, such as two or more types, such as three or more types, such as four or more types, such as five or more types and including ten or more types of bioactive agents. The amount of bioactive agent delivered may be 0.001 mg or more, such as 0.01 mg or more, such as 0.1 mg or more, such as 0.5 mg or more, such as 1 mg or more and including 1 mg or more. For example, the amount of bioactive agent delivered may range from 0.001 mg to 1000 mg, such as 0.01 mg to 500 mg, such as 0.1 mg to 250 mg, such as 0.5 mg to 100 mg, such as 1 mg to 50 mg, including 1 mg to 10 mg

In some embodiments, methods of the present disclosure include using the subject compositions to deliver a dosage of about 0.01 mg/kg to 500 mg/kg of the bioactive agent per day, such as from 0.01 mg/kg to 400 mg/kg per day, such as 0.01 mg/kg to 200 mg/kg per day, such as 0.1 mg/kg to 100 mg/kg per day, such as 0.01 mg/kg to 10 mg/kg per day, such as 0.01 mg/kg to 2 mg/kg per day, including 0.02 mg/kg to 2 mg/kg per day. In other embodiments, methods include using the subject compositions to deliver a dosage of 0.01 to 100 mg/kg four times per day (QID), such as 0.01 to 50 mg/kg QID, such as 0.01 mg/kg to 10 mg/kg QID, such as 0.01 mg/kg to 2 mg/kg QID, such as 0.01 to 0.2 mg/kg QID. In other embodiments, methods include using the subject compositions to deliver a dosage of 0.01 mg/kg to 50 mg/kg three times per day (TID), such as 0.01 mg/kg to 10 mg/kg TID, such as 0.01 mg/kg to 2 mg/kg TID, and including as 0.01 mg/kg to 0.2 mg/kg TID. In yet other embodiments, methods include using the subject compositions to deliver a dosage of may range from 0.01 mg/kg to 100 mg/kg two times per day (BID), such as 0.01 mg/kg to 10 mg/kg BID, such as 0.01 mg/kg to 2 mg/kg BID, including 0.01 mg/kg to 0.2 mg/kg BID.

### Kits

Aspects of the invention further include kits for practicing the methods described herein, where kits include one or more centrifugation containers having a buoy configured to be displaced along a longitudinal axis within the container and a liquid exchange fluid. As described above, in some embodiments, the liquid exchange fluid includes one or more of electrolytes, proteins, activating compounds, ions as well as other active agents. In some embodiments, the liquid exchange fluid includes a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof. In certain embodiments, the liquid exchange fluid is a buffered saline. The liquid exchange fluid in some instances has a citrate ion concentration that is 10 mM or less, such as 5 mM or less, such as 1 mM or less. In certain instance, the liquid exchange fluid does not have any citrate ions (i.e., a citrate ion concentration of 0 mM).

In some embodiments, kits include one or more syringes for introducing the biological sample into the centrifugation container, for removing one or more of the separated fractions, for introducing the liquid exchange fluid and mixing the liquid exchange fluid with a second fraction in the centrifugation container and for removing the liquid exchanged fraction from the container. Where desired, syringes may be configured with a Luer taper fitting, such as a Luer-Lok or a Luer-slip or may be configured with a conduit (e.g., tubing) to fluidly connect one or more syringes to the centrifugation container. Syringes, as well as conduits when present, may also include one or more valves such as a stop-cock valve for controlling the rate of introducing the biological sample or the liquid exchange fluid into the centrifugation container.

In some instances, the kits can include one or more additional components (e.g., buffers, water, solvent etc.). In some instances, the kits may further include a sample collection device, e.g., blood collection device such as an evacuated blood collection tube, needle, syringe, pipette, tourniquet, etc. as desired.

The various assay components of the kits may be present in separate containers, or some or all of them may be pre-combined. For example, in some instances, one or more components of the kit, e.g., centrifugation container, buoys, liquid exchange fluid, syringes, are present in a sealed container or pouch, e.g., a sterile bottle, foil pouch or envelope.

In addition to the above components, the subject kits may further include (in certain embodiments) instructions for assembling the subject kit components as well as for practicing the methods for preparing a liquid exchanged biological sample fraction as described herein. In some embodiments, the instructions included with the kit specify to positioning the centrifugation container at an angle (e.g., from 5° to 45°) with respect to an axis orthogonal to the ground, removing a first fraction of a biological sample from the container, maintaining the centrifugation container at the first angle and introduce the liquid exchange fluid into the container, mixing the liquid exchange fluid with a second fraction of the biological sample in the buoy and removing the liquid exchanged fraction from the container. In some instances, the instructions specify removing 75% by volume or more of the first fraction of the biological sample from the container. In other instances, the instructions specify removing 90% by volume or more of the first fraction of the biological sample from the container.

These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

### Utility

The subject devices, methods and systems find use in a variety of applications where it is desirable to separate components of a multi-component liquid sample and produce a liquid exchanged biological fraction, such as a re-suspended biological fraction, e.g., a cell washed biological fraction, such as described above. Embodiments of the present disclosure also find use in purifying components of a biological sample, such as whole blood and bone marrow aspirate, where it is desirable to obtain isolated components of blood (e.g., white blood cells, stem cells, platelets, etc.) and produced a re-suspended fraction thereof. In some embodiments, the present disclosure finds use in preparing blood products having therapeutic applications, such as re-suspended, e.g., cell washed, platelet rich fractions, e.g., platelet rich saline. Embodiments also find use in the preparation of samples from multi-component liquid where only certain components are desired, such as for laboratory assays, diagnostic tests or for other research applications.

In addition, applications of the present disclosure also find use where components (e.g., cells, proteins, polysaccharides or other large macromolecular compound) prepared from a biological sample may be desired for laboratory testing or for use in therapy. For example, the subject devices and methods facilitate obtaining blood products that may be used to treat wounds, accelerate tissue growth or other ailments.

In some instances, liquid exchanged biological fractions, e.g., as described above, find use in methods of treating a tissue in need of repair in an individual. Aspects of such methods include determining a site of connective tissue injury in the individual; and introducing a liquid exchanged biological fraction, such as platelet-rich saline composition, into and around the site of connective tissue, wherein liquid exchanged biological fraction, e.g., platelet rich saline composition, was prepared from a biologic fluid, e.g., obtained from the individual, using a single centrifugation step, such as described above. In some instances, liquid exchanged biological fractions, e.g., as described above, find use in methods of treating an injured tissue in an individual. In embodiments of such methods, the methods include: determining a site of connective tissue injury in the individual; and introducing a liquid exchanged biological fraction, e.g., platelet-rich composition, into and around the site of connective tissue injury. As the liquid exchanged biological fraction, e.g., platelet-rich composition, is one that is prepared using methods of the invention, e.g., in a method using a single centrifugation step, the concentration of albumin in the composition may be low, such as 3.5 g/dl or less, including 0.35 g/dl or less. In some instances, liquid exchanged biological fractions, e.g., as described above, find use in methods of treating an injured tissue in an individual. In embodiments of such methods, the methods include determining a site of connective tissue injury in the individual; and introducing a liquid exchanged biological fraction, e.g., a platelet-rich composition, into and around the site of connective tissue injury. As the liquid exchanged biological fraction, e.g., platelet-rich composition, is one that is prepared using methods of the invention, e.g., in a method using a single centrifugation step, the concentration of citrate ions in the composition may be low, such as 50 mM or less, such as 10 mM or less and including 1 mM or less. In embodiments of the above therapeutic applications, the initial biological fluid from which the fraction of interest is prepared may vary, and in some instances is peripheral blood, cord blood, or bone marrow aspirate. In some instances, no exogenous activator is added to the liquid exchanged biological fraction, e.g., platelet-rich composition, prior to its introduction into and around the site of injury. In some instances, an exogenous activator is added to the liquid exchanged biological fraction, e.g., platelet-rich composition, prior to its introduction into and around the site of injury. In some instances, fibrinogen is added to the liquid exchanged biological fraction, e.g., platelet-rich composition, prior to its introduction into and around the site of injury. In some instances, hyaluronic acid is added to the liquid exchanged biological fraction, e.g., platelet-rich composition, prior to its introduction into and around the site of injury.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-66 are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method comprising:

introducing a biological sample into a centrifugation container having a buoy configured to be displaced along a longitudinal axis within the container;

subjecting the biological sample to a force of centrifugation to produce two or more fractions in the biological sample;

collecting a first fraction of the biological sample from the container;

introducing a liquid exchange fluid into the container to produce a liquid exchanged fraction comprising a mixture of the liquid exchange fluid and a second fraction of the biological sample; and removing the liquid exchanged fraction from the container.

2. The method according to 1, wherein 75% by volume or more of the first fraction is removed from the container.
3. The method according to 2, wherein 90% by volume or more of the first fraction is removed from the container.
4. The method according to any one of 1-3, wherein the method comprises:

positioning the centrifugation container at an angle with respect to an axis orthogonal to the ground;

removing the first fraction of the biological sample from the container;

maintaining the centrifugation container at the angle and introducing the liquid exchange fluid into the container; and

mixing the liquid exchange fluid with the second fraction of the biological sample in the buoy; and

removing the liquid exchanged fraction from the container.

5. The method according to any one of 1-4, further comprising mixing the liquid exchange fluid with the second fraction in the buoy.
6. The method according to any one of 1-5, further comprising re-introducing the liquid exchanged fraction into the container to rinse the buoy.
7. The method according to any one of 4-6, wherein positioning the centrifugation container at the first angle comprises:

positioning the centrifugation container in an adjustable tilter stand; and

tilting the centrifugation container in the adjustable tilter stand to the angle.

8. The method according to any one of 4-7, wherein the angle is from 5° to 45° with respect to an axis orthogonal to the ground.
9. The method according to any one of 1-8, wherein the biological sample is selected from the group consisting of whole blood, anticoagulated whole blood, bone marrow aspirate, anticoagulated bone marrow aspirate, adipose derived cells, stromal vascular fraction and a combination thereof.
10. The method according to any one of 1-9, wherein the first fraction comprises platelet poor plasma.
11. The method according to any one of 1-10, wherein the second fraction comprises platelets and white blood cells.
12. The method according to any one of 1-11, wherein the liquid exchange fluid is aqueous.
13. The method according to any one of 1-12, wherein the liquid exchange fluid comprises electrolytes.
14. The method according to any one of 1-13, wherein the liquid exchange fluid comprises one or more proteins.
15. The method according to any one of 1-14, wherein the liquid exchange fluid comprises a composition selected from the group consisting of a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof.
16. The method according to 15, wherein the liquid exchange fluid is a buffered saline.
17. A method of producing a liquid exchanged biological fraction of a biological sample using a single centrifugation step.
18. The method according to 17, wherein the method comprises:

introducing a biological sample into a centrifugation container having a buoy configured to be displaced along a longitudinal axis within the container;

centrifuging the centrifugation container to produce multiple fractions of the biological sample, a first fraction of which is positioned above the buoy and second of which is positioned within the buoy;

removing the first fraction from the container;

introducing a liquid, e.g., a cell washing fluid, into the container;

combining the introduced liquid with the second fraction to produce a liquid exchanged second fraction.

19. A method for washing a cell suspension, the method comprising:

introducing a biological sample into a device comprising:
a container comprising a distal end and a proximal end; and
a buoy configured to be displaced along a longitudinal axis within the container;

subjecting the biological sample to a force of centrifugation to produce two or more fractions in the biological sample;

collecting a first fraction or portions of first fraction of the biological sample out of the device;

contacting a second fraction within the buoy with a cell washing fluid;

mixing the cell washing fluid with one or more portions of a second fraction of the biological sample; and

removing the mixture from the container to wash a cell suspension.

20. The method according to 19, wherein collecting one or more components of the biological sample comprises:

removing a portion of a first fraction of the biological sample;

contacting a second fraction within the buoy with a cell washing fluid;

mixing the cell wash fluid and second fraction within the buoy to produce a mixture of the cell wash fluid and the second fraction; and

removing the mixture from the container.

21. The method according to 20, wherein collecting one or more components of the biological sample comprises:

positioning the device at a first angle with respect to an axis orthogonal to the ground;

removing a portion of a first fraction of the biological sample;

rotating the device by a second angle along the longitudinal axis of the container;

aspirating the remaining portion of the first fraction of the biological sample;

contacting a second fraction within the buoy with a cell washing fluid;

mixing the cell wash fluid with the second fraction within the buoy to produce a mixture of the cell wash fluid and second fraction; and

removing the mixture from the container.

22. The method according to 20, wherein collecting one or more components of the biological sample comprises:

positioning the device at a first angular position with respect to an axis orthogonal to the ground;

removing a portion of a first fraction of the biological sample;

contacting a second fraction within the buoy with a cell washing fluid;

tilting the device to first angular position with respect to the axis orthogonal to the ground;

mixing the cell wash fluid with a second fraction within the buoy to produce a mixture of the washing fluid and the second fraction; and

removing the mixture from the container.

23. The method according to any of 20-22, wherein the cell washing fluid is an aqueous fluid.
24. The method according to 23, wherein the aqueous fluid comprises electrolytes.
25. The method according to 23, wherein the aqueous fluid comprises proteins.
26. The method according to any of 20-25, wherein the cell washing fluid is an injectable fluid.
27. The method according to any of 20-26, wherein the first fraction comprises platelet poor plasma.
28. The method according to any of 20-27, wherein the second fraction comprises platelets and white blood cells.
29. The method according to any of 20-28, wherein 90% by volume or more of the first fraction is removed from the container or device.
30. The method according to any of 20-29, wherein the biological sample is selected from the group consisting of anticoagulated whole blood, anticoagulated bone marrow aspirate, and combinations thereof.
31. The method according to any of 20-30, wherein the biological sample is selected from the group consisting of combinations of adipose derived cells and whole blood.
32. The method according to any of 20-30, wherein the second biologic fraction contains viable stem cells.
33. The method according to any of 20-32, wherein the biological fluid is selected from the list consisting of anticoagulated whole blood, anticoagulated bone marrow aspirate, and combinations thereof, wherein 99% by volume or more of the first fraction is removed from the blood sample.
34. The method according to any of 20-33, wherein contacting and mixing the cell washing fluid with the second fraction comprises:

injecting the cell washing fluid using a syringe to contact the second fraction into a syringe;

aspirating the mixture of cell washing fluid and the second fraction into a syringe;

reinjecting the cell washing fluid and the second fraction to rinse the buoy to produce a complete mixing of the cell washing fluid and the second fraction; and

aspirating the mixture from the container with the syringe.

35. A kit comprising:

a liquid exchange fluid; and

a centrifugation container comprising a buoy configured to be displaced along a longitudinal axis within the container.

36. The kit according to 35, further comprising a syringe for removing one or more fractions from the centrifuge container.
37. The kit according to any one of 35-36, wherein the liquid exchange fluid is aqueous.
38. The kit according to any one of 35-37, wherein the liquid exchange fluid comprises electrolytes.
39. The kit according to any one of 35-38, wherein the liquid exchange fluid comprises one or more proteins.
40. The kit according to any one of 35-39, wherein the liquid exchange fluid comprises a composition selected from the group consisting of a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof.
41. The kit according to 40, wherein the liquid exchange fluid is a buffered saline.
42. The kit according to any one of 35-41, further comprising an adjustable tilter stand for positioning the centrifugation container at a plurality of angles with respect to an axis orthogonal to the ground.
43. The kit according to any one of 35-42, further comprising instructions to:

position the centrifugation container at an angle with respect to an axis orthogonal to the ground;

remove a first fraction of a biological sample from the container;

maintain the centrifugation container at the angle and introduce the liquid exchange fluid into the container; and

mix the liquid exchange fluid with a second fraction of the biological sample in the buoy; and

remove the liquid exchanged fraction from the container.

44. The kit according to 43, wherein the kit comprises instructions to remove 75% by volume or more of the first fraction of the biological sample from the container.
45. The kit according to 43, wherein the kit comprises instructions to remove 90% by volume or more of the first fraction of the biological sample from the container.
46. The kit according to any one of 43-45, wherein the kit comprises instructions to position the centrifugation container at an angle of from 5° to 45° with respect to an axis orthogonal to the ground.
47. A composition comprising a liquid exchanged biological fraction.
48. The composition according to 47, wherein the liquid exchange fluid is aqueous.
49. The composition according to any one of 47-48, wherein the liquid exchange fluid comprises electrolytes.
50. The composition according to any one of 47-49, wherein the liquid exchange fluid comprises one or more proteins.
51. The composition according to any one of 47-50, wherein the liquid exchange fluid comprises a composition selected from the group consisting of a saline solution, 0.9% physiologic saline, buffered saline solutions having a pH between 5 and 8, Plasma-Lyte A, Normosol-R, Isolyte-S, lactated Ringers Solution, a hyaluronic acid composition, plasma without anticoagulants, a plasma concentrate fluid, a fibrinogen containing fluid, a drug containing fluid, a cytokine containing fluid, a platelet activating fluid, a thrombin-containing composition, a calcium-containing composition, an ADP-containing composition, a collagen-containing composition and combinations thereof.
52. The composition according to 51, wherein the liquid exchanged biological fraction is a platelet-rich buffered saline.
53. The composition according to any one of 47-52, wherein the liquid exchanged platelet-rich fluid is comprises citrate ions having a concentration of 10 mM or less.
54. The composition according to 53, wherein the liquid exchanged platelet-rich fluid is comprises citrate ions having a concentration of 1 mM or less.
55. The composition according to 53, wherein the liquid exchanged platelet-rich fluid is free of citrate ions.
56. A method of treating a tissue in need of repair in an individual, the method comprising:

determining a site of connective tissue injury in the individual; and

introducing a platelet-rich saline composition into and around the site of connective tissue, wherein the platelet composition was prepared from a biologic fluid using a single centrifugation step.

57. A method of treating an injured tissue in an individual, the method comprising:

determining a site of connective tissue injury in the individual; and

introducing a platelet-rich composition into and around the site of connective tissue injury, wherein the concentration of albumin in the composition is 3.5 g/dl or less and the platelet composition was prepared from a biologic fluid using a single centrifugation step.

58. The method according to 55, wherein the concentration of albumin in the composition is 0.35 g/dl or less.
59. A method of treating an injured tissue in an individual, the method comprising:

determining a site of connective tissue injury in the individual; and

introducing a platelet-rich composition into and around the site of connective tissue injury, wherein the concentration of citrate ions in the composition is 50 mM or less and the platelet composition was prepared from a biologic fluid using a single centrifugation step.

60. The method according to 59, wherein the concentration of citrate ions in the composition is 10 mM or less.
61. The method according to 59, wherein the concentration of citrate ions in the composition is 1 mM or less.
62. The method according to any of 56-61, wherein the biological fluid is selected from the group consisting of peripheral blood, cord blood, and bone marrow aspirate.
63. The method according to any of 56-62, wherein no exogenous activator is added to the composition prior to its introduction into and around the site of injury.
64. The method according to any of 56-62, wherein an exogenous activator is added to the composition prior to its introduction into and around the site of injury.
65. The method of according to any of 56-62, wherein fibrinogen is added to the composition prior to its introduction into and around the site of injury.
66. The method of according to any of 56-62, wherein hyaluronic acid is added to the composition prior to its introduction into and around the site of injury.

### EXPERIMENTAL

The following examples are offered by way of illustration and not by way of limitation. Specifically, the following examples are of specific embodiments for carrying out the present disclosure. The examples are for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

A. Example of cell washing using a buoy containing centrifugation separation device.
1. A Mosaic Platelet Rich Plasma Device cell separator manufactured by MicroAire Surgical containing a buoy with a density of 1.056 was selected for the experiment. Whole blood containing 13% ACD-A was used as the biological fluid. An aliquot of the blood was collected for hematologic analysis as the preprocessing sample to determine cell concentration of red cells, platelets, and white blood cells.
2. 2 ml of ACD-A was injected into the device
3. 60 ml of anticoagulated whole blood was injected into the device
4. The device was then centrifuged for 15 minutes at 1,820×g (3,200 rpm) to stratify the blood.
5. The device was removed from the centrifuge and placed into the titer device with the buoy lined up to position the buoy into the proper position.
6. A 60 ml syringe was attached to the device in the upright position.
7. The device was then tilted to the harvest position of the PRP so as to withdraw all the platelet poor plasma from the device (35 ml) leaving a trace of the platelet poor plasma (>0.1 ml) in the buoy which also contained the platelets, white bloods and some red cells.
8. With the tilter still in the position of PRP harvest, 2 ml of saline was injected into the device port to contact the cell pellet using a 10 ml syringe
9. The 2 ml of fluid and the cell pellet were mixed by pumping the fluid in and out of the syringe a total of 4 times.
10. The resulting platelet rich saline was counted on the hematology analyzer which are summarized in the table below for the preprocessing sample whole blood, platelet poor plasma and the platelet rich saline sample.

| | | | | Mononuclear | |
|---|---|---|---|---|---|
| | | | | (Lymphocytes + | |
| | | | | Monocyes) | |
| Sample | Volume | | Platelet Count | Cell Count | Hematocrit (%) |
| Preprocessing | 60 | ml input | 152 × 10³/ul | 1.5 × 10³/ul | 34 |
| Platelet Poor | 29.4 | ml output | 84 × 10³/ul | 0 × 10³/ul | 0 |
| Plasma | | | | | |
| Platelet Rich | 2.37 | ml output | 3,036 × 10³/ul | 24.6 × 10³/ul | 2.9 |
| Saline | | | (20X baseline) | (16.4X baseline) | |

The above demonstrates the preparation of a platelet concentrate, e.g., platelet rich saline in the above example, from blood or bone marrow without the platelet and white cells being in plasma, where production of the platelet rich saline is achieved with a single centrifugation step. To the knowledge of the inventors, prior to the present invention it was always necessary first to make the PRP and then do a second spin to remove the plasma proteins. The present invention creates opportunities for new formulations of platelet rich solutions that are practical to make in the time available to the clinician since the methods of invention do not require a second spin. Aspects of the invention include the therapeutic use of such platelet concentrates that have been prepared using a single centrifugation step but re-suspended in a fluid reduced in albumin or citrate using methods of invention by exchanging with another fluid as vehicle.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method comprising:
introducing a biological sample into a centrifugation container having a buoy configured to be displaced along a longitudinal axis within the container;
subjecting the biological sample to a force of centrifugation to produce two or more fractions in the biological sample;
collecting a first fraction of the biological sample from the container;
introducing a liquid exchange fluid into the container to produce a liquid exchanged fraction comprising a mixture of the liquid exchange fluid and a second fraction of the biological sample; and
removing the liquid exchanged fraction from the container.

2. The method according to claim 1, further comprising mixing the liquid exchange fluid with the second fraction in the buoy.

3. The method according to claim 1, wherein the method comprises:
positioning the centrifugation container at an angle with respect to an axis orthogonal to the ground;
removing the first fraction of the biological sample from the container;
maintaining the centrifugation container at the angle and introducing the liquid exchange fluid into the container; and
mixing the liquid exchange fluid with the second fraction of the biological sample in the buoy; and
removing the liquid exchanged fraction from the container.

4. The method according to claim 3, further comprising re-introducing the liquid exchanged fraction into the container to rinse the buoy.

5. The method according to claim 3, wherein the first angle is from 5° to 45° with respect to an axis orthogonal to the ground.

6. The method according to claim 1, wherein the biological sample is selected from the group consisting of whole blood, anticoagulated whole blood, bone marrow aspirate, anticoagulated bone marrow aspirate, adipose derived cells, stromal vascular fraction and a combination thereof.

7. The method according to claim 1, wherein the first fraction comprises platelet poor plasma.

8. The method according to claim 1, wherein the second fraction comprises platelets and white blood cells.

9. The method according to claim 1, wherein the liquid exchange fluid comprises buffered saline.

10. A kit comprising:
a liquid exchange fluid; and
a centrifugation container comprising a buoy configured to be displaced along a longitudinal axis within the container.

11. The kit according to claim 10, further comprising an adjustable tilter stand for positioning the centrifugation container at a plurality of angles with respect to an axis orthogonal to the ground.

12. The kit according to claim 10, further comprising instructions to:
position the centrifugation container at an angle with respect to an axis orthogonal to the ground;
remove a first fraction of a biological sample from the container;
maintain the centrifugation container at the angle and introduce the liquid exchange fluid into the container; and
mix the liquid exchange fluid with a second fraction of the biological sample in the buoy; and
remove the liquid exchanged fraction from the container.

13. A composition comprising a liquid exchanged biological fraction.

14. The composition according to claim 13, wherein the liquid exchanged biological fraction is a platelet-rich buffered saline.

15. The composition according to claim 13, wherein the liquid exchanged platelet-rich saline comprises citrate ions having a concentration of 10 mM or less.
